(19) United States
(12) Patent Application Publication
(10) Pub. No.: US 2005/0234545 A1
(43) Pub. Date: Oct. 20, 2005

(54) Amorphous oxide surface film for metallic implantable devices and method for production thereof

(76) Inventors: Yea-Yang Su, Marietta, GA (US); Chun-Che Shih, Taipei (TW); Chun-Ming Shih, Taipei (TW); Shing-Jong Lin, Taipei (TW)

Correspondence Address: MYERS & KAPLAN, INTELLECTUAL PROPERTY LAW, L.L.C., 1899 POWERS FERRY ROAD, SUITE 310, ATLANTA, GA, 30339, US

(21) Appl. No.: 10/827,149

(22) Filed: Apr. 19, 2004

(51) Int. Cl. A61F 2/06

(52) U.S. Cl. 623/1.46; 427/2.25; 428/702

(57) ABSTRACT

An amorphous oxide surface film for metallic implantable devices and method for production thereof, wherein the amorphous oxide film is characterized by a high concentration of oxygen, chromium and hydroxyl ions within the film so as to form a non-stoichiometric chromium oxide with significant negative charge; thereby, improving the corrosion resistance and biocompatibility of the metallic implantable device, and thus significantly reducing the degree of thrombogenicity and restenosis.

### TECHNICAL FIELD

The present invention relates generally to surface treatments and/or coatings for metallic implantable devices, and more specifically to an amorphous oxide surface film for metallic implantable devices and method for production thereof. The present invention is particularly advantageous for its ability to improve corrosion resistance and biocompatibility of metallic implantable stents, and thus, significantly reduce the degree of thrombosis and ensuing restenosis following deployment of same within the coronary artery.

### BACKGROUND OF THE INVENTION

Commonly referred to as “stents”, percutaneously introduced prosthetic devices are utilized to reinforce and maintain the luminal integrity of diseased blood vessels. Of particular recognition and increased usage are intracoronary stents, which have, to date, exhibited increased clinical success due in part to industry manufacturers designing and providing stents specifically manufactured to address the concerns of thrombosis and restenosis.

Specifically, available bare metallic stents characteristically and inherently possess a post-manufacturing oxide layer that tends to interact with the patient's blood and cellular wall following insertion and deployment of the stent within the patient's arteries. Accordingly, poor blood compatibility and/or cellular interaction of the stent with the intimal layer of the artery are dispositive of an adverse interaction, and are typically characterized by increased thrombogenicity and ensuing restenosis. That is, although application of stents have illustrated a reduction in both the incidence of restenosis and the potential of repeat revascularization compared to balloon angioplasty alone, 20% to 40% of patients receiving a stent still experience restenosis as a consequence of adverse stent interaction with the intimal layer of the patients' arteries.

Such adverse stent interaction may be exacerbated as a result of stent corrosion. That is, degradation products, such as metal ions, resulting from corrosion of the metallic stent (i.e., when exposed to physiological conditions) present potential adverse biological effects, namely allergy, cytotoxicity, and carcinogenicity. Additionally, degradation products are well recognized for their proinflammatory effects, as the release of nickel, chromium and molybdenum ions from corroding metallic stents may trigger an immunological defense in the form of chronic inflammatory reactions that would, in turn, result in fibroblast activity and ensuing scar formation. Unfortunately, such inflammatory conditions not only expedite stent corrosion, and thus the release of nickel, chromium and molybdenum ions, but are a direct correlative result of increased thrombogenicity following stent deployment.

Accordingly, several surface treatments and/or coatings have been developed in an attempt to reduce the degree of thrombosis and ensuing restenosis following deployment of the stent into a patient's coronary artery. Such currently available coatings include gold, diamond-like carbon, heparin, silicon, amorphous SiC-H coatings, and drug-eluting coatings such as SIROLIMUS or TAXOL. Unfortunately, each such surface treatment and/or coating, and the methodologies utilized to apply and implement same, possess inherent disadvantages that render application of same overly complex, unduly expensive, and ineffective, especially in view of the generally complex geometry of most available stents.

Specifically, although gold is generally recognized as a highly biocompatible material, stents coated with a gold layer have typically showed no significant influence on the thrombotic event of the stent within the patient's artery, but have illustrated a significant increase in the risk of restenosis through the first year following deployment of the stent. Expensive diamond-like carbon coating of stents is utilized to reduce the release of metallic ions from same, and although clinical application of such stents have illustrated a reduction in neointimal hyperplasia, the degree of reduction is not statistically significant.

With regard to heparin-coated stents, although clinical application of same is characterized by a reduction of platelet deposition, an elimination of cyclic blood flow variation, improved blood flow, and potential reduction of thrombogenicity, such heparin-coated stents have not been shown to improve late vessel patency and neointimal hyperplasia.

Although silicon-based implantable devices are commonly utilized in a variety of medical applications, clinical research has shown that application of impermeable silicon-covered stents could result in increasing thrombogenicity and foreign-body reaction.

With respect to amorphous SIC-H (heavily n-doped hydrogen-rich) coatings, although no clinical trials of such stents have been reported, the coating process is recognizably complex. Typical processes require that the amorphous SIC-H be deposited over the stent surface via complicated plasma-enhanced chemical vapor deposition in an effort to increase the resistance of the surface film and reduce density of electronic states at grain boundaries; thus, reducing electrostatic charging between the stent surface and intimal layer of the artery.

At present, SIROLIMUS is one of the popular drug-eluting coatings utilized on stents, as it is a cell-cycle inhibitor, a natural macrocyclic lactone, and a potent immunosuppressive agent. Accordingly, SIROLIMUS was considered as a coating for stents as it was found during clinical research to inhibit the rate of proliferation of human smooth muscle cells and reduce intimal thickening in a model of vascular injury. Although initial clinical application of SIROLIMUS-coated cardiovascular stents presented no significant clinical events such as stent thrombosis or repeat revascularization, many researchers question whether SIROLIMUS-coated stents are an actual cure to thrombosis and ensuing restenosis, or merely a delay to the occurrence of same.

In addition to, or in lieu of, stent coatings and/or surface treatments, clinical researchers have proposed treatment of the arterial wall via application of radiation utilizing ¹⁹²Iridium seeds in an attempt to reduce neointima formation following balloon angioplasty, and just prior to stent deployment. Unfortunately, however, irradiation of non-target tissue surrounding the arterial wall, in addition to exposure of the operating person to the radiation, renders such treatment highly risky, especially in view of the fact that a relatively high threshold of radiation (approximately 4 μCi) must be delivered and maintained to inhibit neointima formation. However, even with such radiation treatment, and following implantation of the stent, it is shown that neointima thickening still occurs with implant time.

Accordingly, in an attempt to resolve the above-discussed disadvantages, much research has been conducted with regard to the post-manufacturing oxide layer that exists over bare metallic stents, and the interaction of same with the patient's intima. Specifically, because a surface oxide film is a semiconductor for electron transfer during the thrombosis process, it is recognized that the creation of more negatively-charged surface oxide could reduce thrombogenicity. Such a negatively-charged oxide surface may be achieved via metal passivation techniques conducted under controlled conditions, wherein such passivated metals tend to exhibit a much more stable oxide layer over their surface; thus, significantly contributing to corrosion resistance and relative inertness of the metallic stent when deployed in physiological conditions.

Of the various oxide layers commonly encountered over metallic stents, stents comprising polycrystalline oxide layers are recognized as being generally unsuitable for cardiovascular application, due in large part to the relatively high density of state in the band gap in the grain boundaries (i.e., oxide particle boundaries), wherein such a high density of state fails to satisfy the necessity of a low transfer current density for reduced thrombogenicity. That is, a lower density of state is held to be a critical factor in determining the degree of thrombosis and restenosis following implantation of the stent.

Although, electropolishing and nitric acid passivation are techniques currently utilized to create desired protective oxide films over the stent surface for increased corrosion resistance and improved biocompatibility of same, such techniques still do not provide the requisite density of state of oxide particles or grains over the surface of treated metallic stent.

Therefore, it is readily apparent that there is a need for an amorphous oxide surface film for metallic implantable devices and method for production thereof, wherein the present invention may be utilized to manufacture a stent comprising corrosion resistance and enhanced biocompatibility, thereby significantly reducing the degree of thrombosis and ensuing restenosis following deployment of same within a physiological environment.

### BRIEF SUMMARY OF THE INVENTION

Briefly described, in a preferred embodiment, the present invention overcomes the above-mentioned disadvantages and meets the recognized need for such an apparatus and method by providing an amorphous oxide surface film for metallic implantable devices and method for production thereof, wherein the amorphous oxide film comprises a high concentration of oxygen, chromium and hydroxyl ions within the film, so as to form a non-stoichiometric chromium oxide with significant negative charge; thereby, improving the corrosion resistance and biocompatibility of the metallic implantable device, and thus significantly reducing the degree of thrombogenicity and restenosis.

According to its major aspects and broadly stated, the present invention in its preferred form is an amorphous oxide surface film for metallic implantable devices and method for production thereof, comprising, in general, passivation processes and solutions utilizing sodium nitrate (NaNO₃) as a preferred oxygen provider to facilitate formation of an amorphous oxide surface film over a selected implantable device.

More specifically, the present invention is an amorphous oxide surface film for metallic implantable devices and method for production thereof, wherein an implant sample is preferably heated to an appropriate temperature within a saturated oxygen atmosphere such that the nanometer or sub-nanometer scale of amorphous oxide particles form at a faster rate of nucleation than growth; thus, resulting in an amorphous oxide film comprising a high concentration of oxygen, chromium and hydroxyl ions therewithin, and, as such, a non-stoichiometric chromium oxide with significant negative charge.

Preferably, the formation of the present amorphous oxide film may be selectively obtained from one of either three passivation solutions (i.e., referred to herein as passivation solutions A, B and C) depending on the final application or end-use of the implants, as some implants, and the application thereof, favor an oxide film formed in an acidic solution, while others favor an amorphous oxide film formed in either an alkaline solution or a neutral solution.

Accordingly, passivation solutions A, B, and C preferably utilize sodium nitrate (NaNO₃) as the oxygen provider to facilitate formation of the present amorphous oxide layer. However, it should be recognized that other suitable nitrate compounds may be utilized in substitution of NaNO₃ to form the amorphous oxide film of the present invention over the selected implant, wherein such alternate nitrate compounds may include, without limitation and for exemplary purposes only, potassium nitrate, ammonium nitrate, calcium nitrate, chromium nitrate, copper nitrate, iron nitrate, lead nitrate, and barium nitrate.

With specific regard to passivation solution A, preferred pH buffer chemicals include a combination of sodium bicarbonate (NaHCO₃), sodium carbonate (Na₂CO₃), and sodium hydroxide (NaOH), as such buffers function not only as good pH buffers at elevated temperatures, but further behave as oxygen donors at the processing temperature. It is contemplated, however, that the preferred pH buffer chemicals may be replaced with other chemicals, such as, for exemplary purposes only, phosphate or borate compounds, if no intervention of formation of the present amorphous oxide film occurs. Preferably, the ratio between NaHCO₃, Na₂CO₃, and NaOH is 1:1:1 for most applications, but may be adjusted to any ratio or as high as 1:1:10, or even 1:1:20, if a higher concentration of hydroxyl ion is required for the amorphous oxide film—such as in those circumstances wherein the amorphous oxide layer will be utilized as a platform for drug-loading.

Although passivation solution A provides a higher concentration of hydroxyl ions within the resulting amorphous oxide layer than passivation solution B, which provides little to no hydroxyl ions within the amorphous oxide film as a result of the lower pH value of passivation solution B, amorphous oxide layers resulting from utilization of passivation solution B still preferably impart the implants with improved physical, chemical, and biocompatibly favorable properties in view of traditional implant coatings and/or surface treatments.

With regard to passivation solution C, depending on the chemical composition of the alloys of the metallic implantable device, the pH value of passivation solution C (preferably comprising NaNO₃) may be selectively adjusted to the range of approximately 6.5 to approximately 7.5 to effectively enhance the corrosion resistance of such metal-alloy devices. Accordingly, and preferably with the assistance of a pH meter, neutral passivation solution C is preferably imparted with such a pH value by adjusting and titrating same via the addition of preferably small amounts of NaHCO₃ and diluted HCl solution.

Preferably, the processing temperature for each passivation solution A, B or C must reach at least the boiling temperature of the respective solution in order to provide the highest density of oxygen ion concentration inside the resulting amorphous oxide surface film. It should be noted that the processing time may be reduced with higher processing temperatures.

Preferably, to maintain the proper concentration of the various chemical components within passivation solutions A, B or C during the amorphous oxide formation processing, a condenser with running water circulating therearound is preferably utilized. Additionally, the glass container/flask utilized in implementing the present process may be replaced with other suitable containers of proper materials adapted to effectively handle the heat transfer of the present invention, and provide effective corrosion resistance to the high pH, low pH, or neutral pH of passivation solutions A, B or C, respectively.

The present invention contemplates that the amorphous oxide film processing preferably be performed as a final step in the manufacturing process of the implant; although, application of the present method may be utilized at any selected step of the manufacturing process to yield desired results.

Accordingly, a feature and advantage of the present invention is its ability to provide an implantable device comprising an amorphous oxide surface film that provides the implantable device with excellent corrosion resistance to chloride-bearing solutions, such as body fluid, or tissue comprising high concentrations of chloride.

Another feature and advantage of the present invention is its ability to provide an implantable device comprising an amorphous oxide surface film that comprises a negative and stable open-circuit potential when exposed to body fluid or tissue; thus, ensuring a thrombosis-free condition.

Still another feature and advantage of the present invention is its ability to provide an implantable device comprising an amorphous oxide surface film having a negative charge thereover; thus, inhibiting the release of positively-charged ions from the implantable device.

Still yet another feature and advantage of the present invention is its ability to provide a stent comprising an amorphous oxide surface film, wherein the high value of time constant over the amorphous oxide surface film functions to retard the interaction between the stent and blood and/or intimal layer of an artery; thus, reducing thrombogenicity and ensuing restenosis.

A further feature and advantage of the present invention is its ability to provide a stent comprising an amorphous oxide surface film that functions to effectively minimize restenosis following deployment of same with a selected artery.

Still a further feature and advantage of the present invention is its ability to provide an implantable device comprising an amorphous oxide surface film that effectively provides and functions as a platform for drug-loading, wherein such drug-loading may advantageously be effectuated without the assistance of a polymer as a result of the chemical and structural configuration of the present amorphous oxide layer.

These and other features and advantages of the present invention will become more apparent to one skilled in the art from the following description and claims when read in light of the accompanying drawings.

### BRIEF DESCRIPTION OF THE DRAWINGS

The present invention will be better understood by reading the Detailed Description of the Preferred and Alternate Embodiments with reference to the accompanying drawing figures, in which like reference numerals denote similar structure and refer to like elements throughout, and in which:

FIG. 1 is a flow diagram of a method of amorphous oxide surface film formation according to a preferred embodiment of the present invention;

FIG. 2 is an illustration of apparatus utilized to implement a method of amorphous oxide surface film formation according to a preferred embodiment of the present invention;

FIG. 3 is an image of transmission electron microscopy of various oxide layers, including an amorphous oxide surface film according to a preferred embodiment of the present invention, observed at low magnification;

FIG. 4 is an image of transmission electron microscopy of an amorphous oxide surface film according to a preferred embodiment of the present invention, observed at high magnification;

FIG. 5A is a graphical diagrammatic representation of oxygen and chromium concentrations of an amorphous oxide surface film according to a preferred embodiment of the present invention;

FIG. 5B is a graphical diagrammatic representation of oxygen and chromium concentrations of an oxide film yielded through electropolishing processes;

FIG. 5C is a graphical diagrammatic representation of oxygen and chromium concentrations of a polycrystalline oxide film;

FIG. 6 is a graphical diagrammatic representation of open circuit potential measurements of various oxide layers, including an amorphous oxide surface film according to a preferred embodiment of the present invention;

FIG. 7 is a graphical diagrammatic representation of cyclic anodic polarization scanning curves of various oxide layers, including an amorphous oxide surface film according to a preferred embodiment of the present invention;

FIG. 8A is a graphical diagrammatic representation of current density at open-circuit potential for an amorphous oxide surface film according to a preferred embodiment of the present invention;

FIG. 8B is a graphical diagrammatic representation of current density at open-circuit potential for an oxide film yielded through electropolishing processes;

FIG. 8C is a graphical diagrammatic representation of current density at open-circuit potential for a polycrystalline oxide film;

FIG. 9 is a graphical diagrammatic representation of time constant versus degree of thrombosis for various oxide layers, including an amorphous oxide surface film according to a preferred embodiment of the present invention;

FIG. 10A is an image of a heparin-medicated implant comprising an amorphous oxide surface film according to a preferred embodiment of the present invention, illustrating degree of thrombosis;

FIG. 10B is an image of a heparin-medicated implant comprising an oxide film yielded through electropolishing processes, illustrating degree of thrombosis;

FIG. 11A is an image of an implant comprising an amorphous oxide surface film according to a preferred embodiment of the present invention, illustrating degree of neointimal hyperplasia;

FIG. 11B is an image of an implant comprising an oxide film yielded through electropolishing processes, illustrating degree of neointimal hyperplasia;

FIG. 11C is an image of an implant comprising a polycrystalline oxide film, illustrating degree of neointimal hyperplasia; and,

FIG. 12 is a graphical diagrammatic representation of cyclic voltammetry for an amorphous oxide surface film according to a preferred embodiment of the present invention, utilized as a platform for drug loading.

### DETAILED DESCRIPTION OF THE PREFERRED AND SELECTED ALTERNATIVE EMBODIMENTS

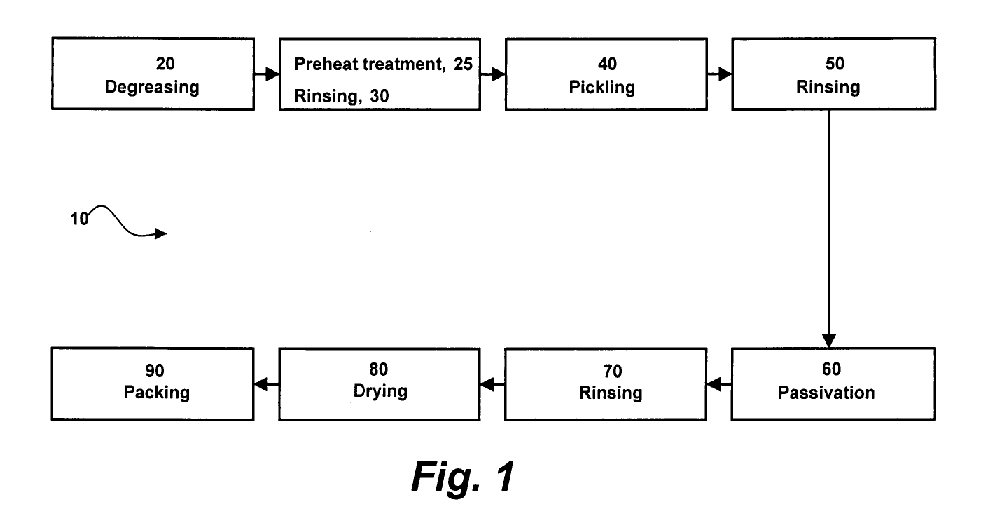

In describing the preferred and selected alternate embodiments of the present invention, as illustrated in FIGS. 1-12, specific terminology is employed for the sake of clarity. The invention, however, is not intended to be limited to the specific terminology so selected, and it is to be understood that each specific element includes all technical equivalents that operate in a similar manner to accomplish similar functions.

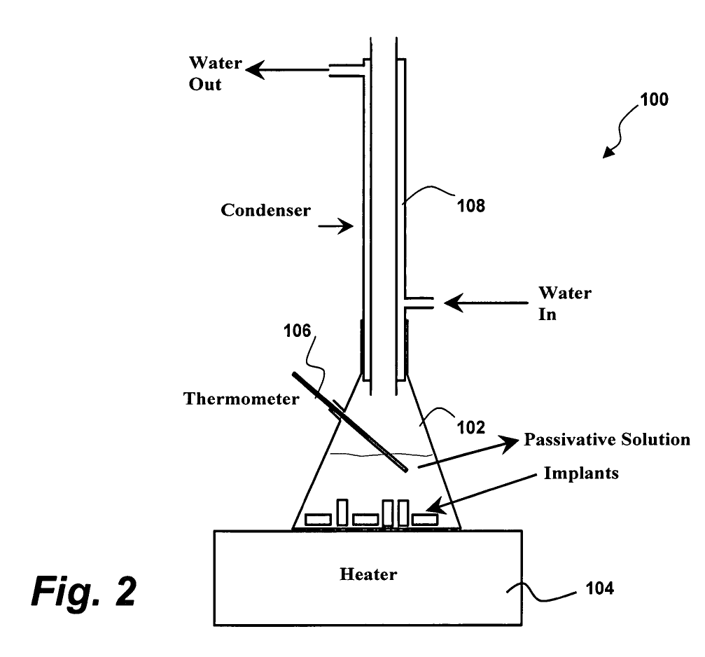

Referring specifically now to FIGS. 1-2, the present invention in a preferred embodiment is an amorphous oxide surface film 5 for metallic implantable devices, and method 10 for production thereof, wherein method 10 comprises the steps of degreasing 20, pre-heat treatment 25, rinsing 30, pickling 40, rinsing 50, passivation 60, rinsing 70, drying 80, and packing 90. As more fully described below, passivation step 60 of method 10 is preferably implemented via apparatus 100, wherein apparatus 100 preferably comprises flask 102, heater 104, thermometer 106 and condenser 108. Although passivation step 60 of method 10 is preferably implemented via apparatus 100, it should be recognized that other suitable apparatuses, assemblies and/or equipment may be utilized to effectuate passivation step 60 to effectively yield amorphous oxide surface film 5 of the present invention.

Preferably, the present invention contemplates that amorphous oxide surface film 5 may be formed over any selected implantable device, and more specifically, over any implantable device manufactured from a suitable material, such as, for exemplary purposes only, stainless steels (including, without limitation stainless steel 316, 316L, 316LVM, 316LN, 304, and/or 304L), MP35N alloy, Chromium-Cobalt alloys, NITINOL, Titanium, Ti-6Al-4V (i.e., titanium alloy), or Zirconium. Additionally, the present invention further contemplates that suitable implantable devices preferably include, without limitation, wire, stents, grafting, and/or other implants of any selected geometric shape.

Preferably, method 10 begins with degreasing step 20, wherein all as-manufactured implants, including stents, strips, disks, or the like, are preferably degreased with a suitable solvent, such as, for exemplary purposes only, isopropyl alcohol or trichloroethylene, in order to remove post-manufacturing lubricants or other oily materials from the surfaces thereof.

Following thorough degreasing of the implants via degreasing step 20, all implants preferably undergo pre-heat treatment step 25 to ensure a high quality amorphous oxide surface film 5. Specifically, during pre-heat treatment step 25, all implants are preferably heat-treated at approximately 800° C., wherein stents are heated for at least two minutes, strips for at least five minutes, and disks for at least ten minutes; however, it should be recognized that the actual time for heat treatment is a function of the thickness of the implants/samples.

Preferably, the purpose of pre-heat treatment step 25 is to homogenize all heterogeneous structures within the implants, as such heterogeneous structures could create galvanic current inside the final amorphous oxide surface film 5, and therefore, adversely affect the otherwise various advantageous physical and chemical properties. If, however, the selected implants comprise an appropriate crystalline structure, pre-heat treatment step 25 may be selectively omitted.

Following pre-heat treatment step 25, and subsequent cooling of the implants, all implants are preferably cleansed or rinsed 30 via running water.

Thereafter, the implants preferably undergo pickling step 40, wherein pickling step 40 is preferably utilized to remove the oxide film that inherently resides over post-manufactured implants, and/or the materials utilized to manufacture the implant. Accordingly, such pre-existing oxide films are preferably removed via a pickling solution comprising a first solution of approximately 10 cc of hydrofluoric acid (concentrated) mixed with approximately 40 cc of distilled water, and a second solution of approximately 25 cc nitric acid (concentrated) mixed with approximately 25 cc distilled water, wherein the first and second solutions are preferably mixed together at a ratio of approximately 1:1 to make the requisite pickling solution for utilization in pickling step 40. The preferred pickling solution is preferably a recognized industrial and ASTM standard pickling solution typically utilized to remove surface oxide films on stainless steel implants; however, it should be recognized that other suitable pickling solutions may be utilized for stainless steel implants and/or implants of any other selected metal or metal alloy.

Utilizing the above-described pickling solution, all implants are preferably ultrasonically pickled for approximately five to seventy minutes depending on the oxide film thickness over the implants.

Following pickling step 40, all implants are preferably cleansed or rinsed 50 via running water.

Thereafter, the implants are preferably introduced into preferred passivation solutions A, B or C via passivation step 60; thereby, resulting in the formation of amorphous oxide surface film 5 thereover. Preferably, passivation step 60 is conducted via utilization of apparatus 100, as more fully described below.

Generally, passivation solutions A, B and C preferably utilize sodium nitrate (NaNO₃) as the oxygen provider to facilitate formation of the present amorphous oxide surface film 5. However, it should be recognized that other suitable nitrate compounds may be utilized in substitution of NaNO₃ to form amorphous oxide surface film 5 of the present invention over the selected implant, wherein such alternate nitrate compounds may include, without limitation and for exemplary purposes only, potassium nitrate, ammonium nitrate, calcium nitrate, chromium nitrate, copper nitrate, iron nitrate, lead nitrate, and barium nitrate.

With specific regard to passivation solution A, preferred pH buffer chemicals include a combination of sodium bicarbonate (NaHCO₃), sodium carbonate (Na₂CO₃), and sodium hydroxide (NaOH), as such buffers function not only as good pH buffers at elevated temperatures, but further behave as oxygen donors at the processing temperature. It is contemplated, however, that the preferred pH buffer chemicals may be replaced with other chemicals, such as, for exemplary purposes only, phosphate or borate compounds, if no intervention of formation of the present amorphous oxide surface film 5 occurs. Preferably, the ratio between NaHCO₃, Na₂CO₃, and NaOH is approximately 1:1:1 for most applications, but may be adjusted to any ratio or as high as approximately 1:1:10, or even approximately 1:1:20, if a higher concentration of hydroxyl ion is required for amorphous oxide surface film 5—such as in those circumstances wherein amorphous oxide surface film 5 will be utilized as a platform for drug-loading.

Accordingly, passivation solution A is preferably manufactured and utilized to impart amorphous oxide surface film 5 over the implants as follows: (1) approximately 100 cc of distilled water is preferably mixed with approximately 1.5 g of NaHCO₃, approximately 1.5 g Na₂CO₃, and approximately 1.5 g of NaOH; (2) the resulting mixture is preferably agitated until all the ingredients are dissolved to yield a solution comprising a pH of approximately around or higher than 10; (3) approximately 50 cc of the solution is preferably added to flask 102 of apparatus 100; (4) approximately 50 g of NaNO₃ is preferably added to the solution within flask 102; (5) the mixture within flask 102 is preferably heated, via heater 104 of apparatus 100, until the whole solution is boiling, whereupon the implants are then added to the boiling solution (i.e., boiling temperature is approximately around 125° C., and may be measured via thermometer 106 of apparatus 100), and wherein bringing the solution to boiling temperature preferably provides the highest density of oxygen ion concentration inside the resulting amorphous oxide surface film 5; and, (6) to maintain the proper concentration of the various chemical components within passivation solution A, condenser 108 of apparatus 100 is preferably utilized in conjunction with flask 102, wherein running water is preferably circulated through condenser 108.

Preferably, the optimal composition of passivation solution A approximately includes 1000 g/l of NaNO₃, 15 g/l of NaHCO₃, 15 g/l of Na₂CO₃, and 15 g/l of NaOH. However, depending upon the application of the implant and the desired properties of amorphous oxide surface film 5 thereover, the composition of passivation solution A may approximately include ranges of 10-2000 g/l of NaNO₃, 0.1-50 g/l of NaHCO₃, 0.1-50 g/l of Na₂CO₃, and 0.1-50 g/l of NaOH.

Although passivation solution A provides a higher concentration of hydroxyl ions within the resulting amorphous oxide surface film 5 than passivation solution B, which provides little to no hydroxyl ions within amorphous oxide surface film 5 as a result of the lower pH value of passivation solution B, amorphous oxide layers resulting from utilization of passivation solution B still preferably impart passivated implants with improved physical, chemical, and biocompatibly favorable properties in view of traditional implant coatings and/or surface treatments.

Accordingly, passivation solution B is preferably manufactured and utilized to impart amorphous oxide surface film 5 over the implants as follows: (1) approximately 50 cc of water (distilled or de-ionized water is preferred) is preferably added to flask 102 of apparatus 100; (2) to the water, approximately 50 g of NaNO₃ is preferably added; (3) preferably, the pH of the resulting solution is diluted to a value of approximately 2 or lower with a HNO₃ solution comprising an approximately 1:1 ratio of concentrated HNO₃ to water; (4) the entire solution within flask 102 is preferably heated, via heater 104, until boiling, whereupon the implants are then added to the boiling solution (i.e., boiling temperature is approximately around 125° C., and may be measured via thermometer 106 of apparatus 100), and wherein bringing the solution to boiling temperature preferably also provides the highest density of oxygen ion concentration inside the resulting amorphous oxide surface film 5; and, (5) to maintain the proper concentration of the various chemical components within passivation solution B, condenser 108 of apparatus 100 is preferably utilized in conjunction with flask 102, wherein running water is preferably circulated through condenser 108.

Preferably, the optimal composition of passivation solution B approximately includes 1000 g/l of NaNO₃; however, depending upon the application of the implant and the desired properties of amorphous oxide surface film 5 thereover, the composition of passivation solution B may approximately include a range of 10-2000 g/l of NaNO₃.

With regard to passivation solution C, depending on the chemical composition of the alloys of the metallic implantable device, the pH value of passivation solution C (preferably comprising NaNO₃) may be selectively adjusted to the range of approximately 6.5 to approximately 7.5 to effectively enhance the corrosion resistance of such metal-alloy devices. Accordingly, and preferably with the assistance of a pH meter, neutral passivation solution C is preferably imparted with such a pH value by adjusting and titrating same via the addition of preferably small amounts of NaHCO₃ and diluted HCl solution, wherein equivalent methodologies utilized to manufacture passivation solutions A and B, as described above, may be utilized to manufacture neutral passivation solution C.

For any selected passivation solution A, B or C, passivation time of the implants is preferably conducted for a at least thirty minutes for most implants, and may be conducted for up to two hours for other applicable implants following achievement of boiling temperature of the respective solutions; however any suitable passivation time may be utilized during passivation step 60 to yield desired results.

Following passivation step 60, all implants are preferably cleansed or rinsed 70 via running water. Thereafter, the implants are preferably dried 80 via circulated cool air, and subsequently packaged 90. Heated air is preferably not utilized to dry the implants following rinsing step 70.

Prior to packing 90, sterilization of the implants may be implemented via introduction of the dried implants to gamma rays, ethylene oxide, and/or 70% alcohol, especially if the implants undergoing sterilization are for subsequent cardiovascular application. It should be noted that steam sterilization is not a preferred method of sterilization, as such a process could transfer/change amorphous oxide surface film 5 into a polycrystalline oxide; thus, destroying the significant electrochemical characteristics of amorphous oxide surface film 5.

To measure the effectiveness of amorphous oxide surface film 5 and/or implant comprising amorphous oxide surface film 5 thereover, various properties of amorphous oxide surface film 5 may be characterized in vitro by the following experimental techniques:

Anodic polarization measurement: Corrosion resistance of different surface oxide films on stainless steel or other implants can be evaluated by cyclic anodic polarization measurement. The cyclic anodic polarization is implemented via a computer-controlled potentiostat (Ex.: EG&G Princeton Applied Research, Model 273). Tests are conducted at 37° C. in Ringer's physiological solution with the following composition: NaCl: 9.0 g/l, CaCl₂*2H₂O:0.17 g/l; KCl: 0.4 g/l, wherein the solution is buffered with NaHCO₃ with a concentration of 2.1 g/l and maintained at the normal physiological pH of 7.4. Saturated calomel electrode (SCE) is utilized as the reference and a platinum wire is utilized as the counter electrode. The solution is continuously purged with 5% CO₂/95% air of mixed gas for 1 hour before starting and during the measurement. A scan rate of 0.167 mV/s is applied starting at −0.15 Vvs. SCE is utilized until a breakdown potential is reached, and potential subsequently reverses. Experimentation is terminated once the reversal scanning potential reaches repassivation potential.

Open-circuit potential (OCP): Open-circuit potential (OCP) of each passivated surface is measured with respect to the standard calomel electrode (SCE) using computer-controlled potentiostat (Ex.: EG&G Princeton Applied Research, Model 273) in aerated Ringer's solution at 37° C. OCP is a critical factor for the formation of thrombosis after implants are deployed into the artery.

Current density at open-circuit potential: Current density at OCP is measured for stainless steel with various surface conditions, and is recorded as a function of time. This electrochemical measurement is designed to investigate the stability of the passivated oxide film and the possibility of releasing positively-charged ions when the implant contacts with body fluid or Ringer's solution.

Transmission electron microscopy (TEM): Characterization of various oxide films are performed by transmission electron microscopy (TEM). The thin oxide film is stripped from the substrate by dissolving the oxidized stainless steel into a 10% bromine-methanol solution at 40° C. for thirty minutes, wherein the thin oxide films are retained on the filter paper after the whole solution is filtered. The retained oxide films are cleaned thoroughly by methanol and then transferred to a copper mesh for TEM examination. Structure is investigated by bright field and selected area diffraction (SAD).

Scanning electron microscopy (SEM): Surface morphology of an implant is examined by scanning electron spectroscopy to determine the severity of corrosion or degree of thrombosis. Representative micrographs are taken in a secondary electron imaging mode. To prevent charging problems and enhance the resolution, implants are sputtered with a thin layer of gold.

Auger electron spectroscopy (AES): AES analysis is performed on the passivated surfaces (670 PHI Xᵢ, Physical Electronics, USA) at 5 keV with a 9.8 nA primary electron beam. Ion etching is performed at a pressure of 10 mPa using high purity argon and a raster size 4×4 mm², with the corresponding sputtering rate at 2.7 nm/min calibrated by SiO₂. The main peaks utilized for determination of the atomic percentage in the depth profiles are O KLL at 510 eV, Cr LMM at 49 eV, Fe LMM at 589 eV, Mo MNN at 184 eV, and Ni LMM at 844 eV. The atomic percent is calculated from the peak areas with software equipped with AES. Depth profiles are measured by combining AES analysis and argon ion sputter etching to evaluate the oxide layer thickness and the elemental distribution.

Cyclic voltammetry (CV): CV analysis is carried out utilizing the EG&G Potentiostat Model 273 in conjunction with the software Model 253 Version 4.1.1. CV tracings are recorded in aerated Ringer's solution at 37° C. from −900 mV to +400 mV, at a rate of 20 mV/sec versus a saturated calomel electrode (SCE) reference electrode. A three-electrode system is utilized throughout the study. The working electrode is drug-coated stainless steel implant, wherein a platinum wire serves as a counter electrode.

Electrochemical Impedance Spectroscopy (EIS): An AC impedance measurement technique is employed to investigate the electrochemical kinetics at the implant-electrolyte interface. The measurement is performed at an open-circuit potential and the frequency is varied in the range of 10⁵ Hz to 10⁻⁵ Hz with an imposed voltage of 5 mV AC (i.e., utilizing Model 6310, EG &G, USA).

The above-discussed in vitro characterizations or experiments preferably allow the detection of the properties of amorphous oxide surface film 5, and other selected oxide films, when submerged in an electrolyte solution, wherein parameters such as capacitance and resistance are generally recorded. Since the thin amorphous oxide surface film 5 acts as a capacitor when the semiconductor device makes contact with physiological fluids or blood, the time it takes for the reaction to take place is an important characteristic of each type of amorphous oxide surface film 5. The time constant, A, is calculated by the multiplication of the values of capacitance and resistance determined from the impedance measurements.

In addition to in vitro characterization of amorphous oxide surface film 5 and/or implants comprising amorphous oxide surface film 5, in vivo characterization is also preferably conducted via the following experimental techniques:

Animal Experiment: The potential effect of metal surface treatments on post-stenting neointimal formation is evaluated in abdominal aorta of New-Zealand white rabbits, wherein adult male New-Zealand white rabbits weighing 3.12±0.08 kg (2.47-3.52 kg), and fed with normal diet, are utilized in this study. The New Zealand rabbits are anesthetized by intra-muscular injection of ketamine (50 mg/kg) mixed with xylazine (10 mg/kg). Femoral artery was exposed and ligated. A 5 Fr. introducer sheath is passed via arteriotomy retrograde into the abdominal aorta with systemic heparinization (100 USP units/kg) intra-arterially. Balloon catheters are subsequently introduced through this sheath and advanced proximally to the aortic arch via a 0.014 inch guide wire. The stainless steel stents are delivered by an angioplasty balloon catheter. Each stent is then deployed in the lower abdominal aorta by two consecutive balloon inflation pressures of 40 seconds at 8 atm to a final diameter of 4 mm. The infrarenal abdominal aorta is approximately 3.7 mm in diameter yielding a stent/artery ratio of 1.1:1 to 1.2:1. The punctured artery is ligated both proximally and distally. Rabbits received ampicillin immediately after operation.

Morphometric Measurement: Cross-section of neointimal and media surface area is determined utilizing computer-assisted digital planimetrys. The intimal area and media area are determined at proximal, middle, and distal site from each stent, and the results are averaged to minimized sampling error.

Tissue Processing: Stented abdominal aortas are harvested after 8 weeks. The harvested stents are fixed by immersion in 4% paraformaldehyde. Stented arterial segments are oriented from distal to proximal end. The whole segment is dehydrated step wisely with graded alcohols. Specimens are then embedded in epoxy-araldite resin. Multiple stented aortas are serially sectioned from one end to the middle portion of the segment, wherein five even slides per stent from the total 20 sections of each animal are produced. Sectioning is performed by a rotating diamond-coated saw, with the stent struts remaining in situ. The thickness of each slice is about 100-150 μm. Slices are stained with toluidine blue to enhance the areas of media and intima for observation and histology calculation. The extent of deep arterial injury caused by stent struts is quantified histologically in Verhoeff elastin-stained or hematoxylin and eosin.

Thrombosis Study via Experimental Model: Experiments are performed on mongrel dogs with a mean body weight of 17.2±1.6 kg (range, 15.6-18.5 kg). After overnight fasting, dogs are sedated with Phenobarbital (5 mg/kg), and anesthesia is maintained with 1.5% halothane after endotracheal intubation. Both arterial and venous limbs of fistula are isolated and implanted with stainless steel stents having different surface oxides. Arterial blood gases and pH are monitored periodically and maintained at normal levels by adjusting ventilation rate and tidal volume. Intensive arterial pressure measurement, oxygen saturation, ECG, and rectal temperature are monitored continuously. A thermostatically controlled blanket is utilized to maintain temperature at 37° C.

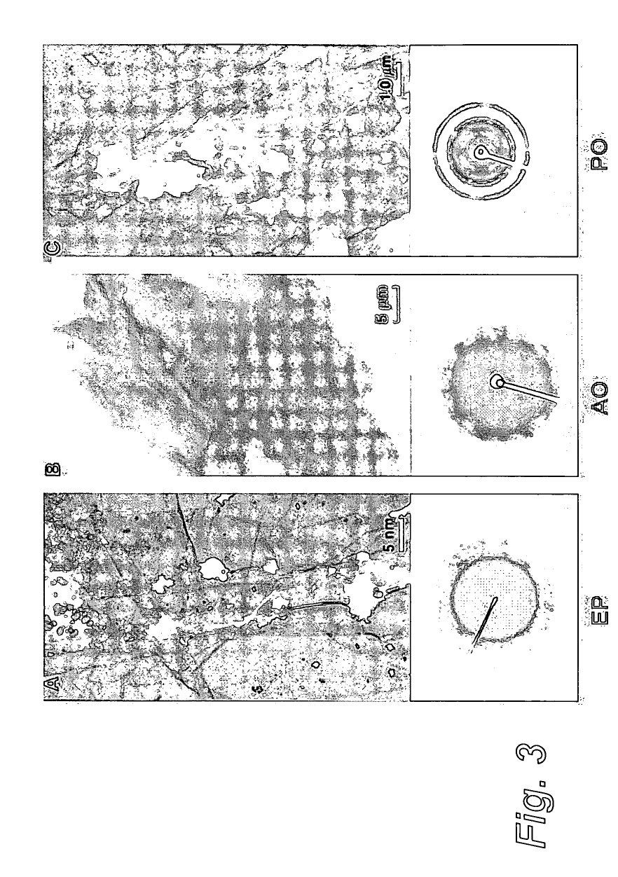

Referring now more specifically to FIGS. 3-12, and with reference to the above-discussed in vitro and/or in vivo characterization techniques and experiments, properties of implants comprising amorphous oxide surface film 5 (referred to hereinafter as “AO”) may be distinguished from those implants comprising an oxide film created via electropolishing processes (referred to hereinafter as “EP”), and implants comprising a polycrystalline oxide film (referred to hereinafter as “PO”).

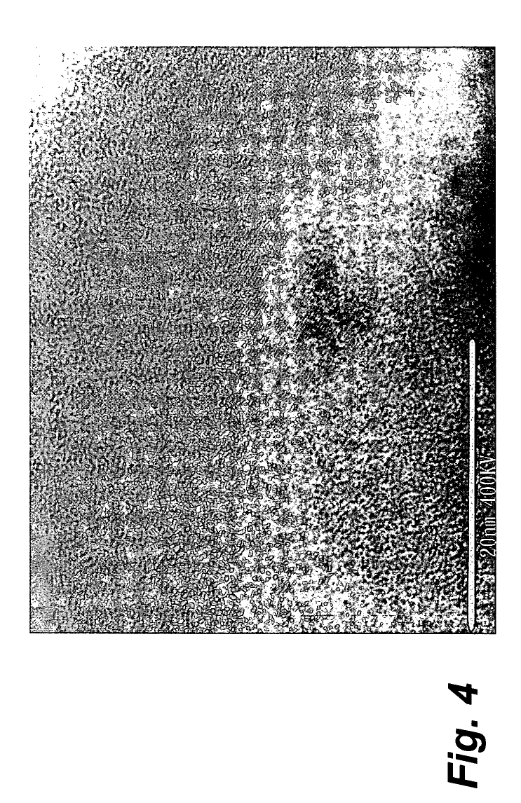

With specific reference to FIGS. 3-4, oxide structures and particles of AO, EP and PO films may be examined via transmission electron microscopy after proper sample preparation, as described above. Further, crystal structures may be determined by the selected area diffraction (SAD) pattern. Accordingly, FIGS. 3-4 illustrate that the oxide film created by EP comprises oxide grains in nanometer-scale to micro scale range, as is confirmed by defined rings revealed by the SAD. Oxide particles are in the wide ranges for the PO films.

Unlike the EP and PO films, the diffused rings observed from the SAD for the stainless steel implant coated with AO indicate that oxide particles are in nanometer scale or sub-nanometer scale within amorphous oxide surface film 5, as is shown in the higher magnification of FIG. 4.

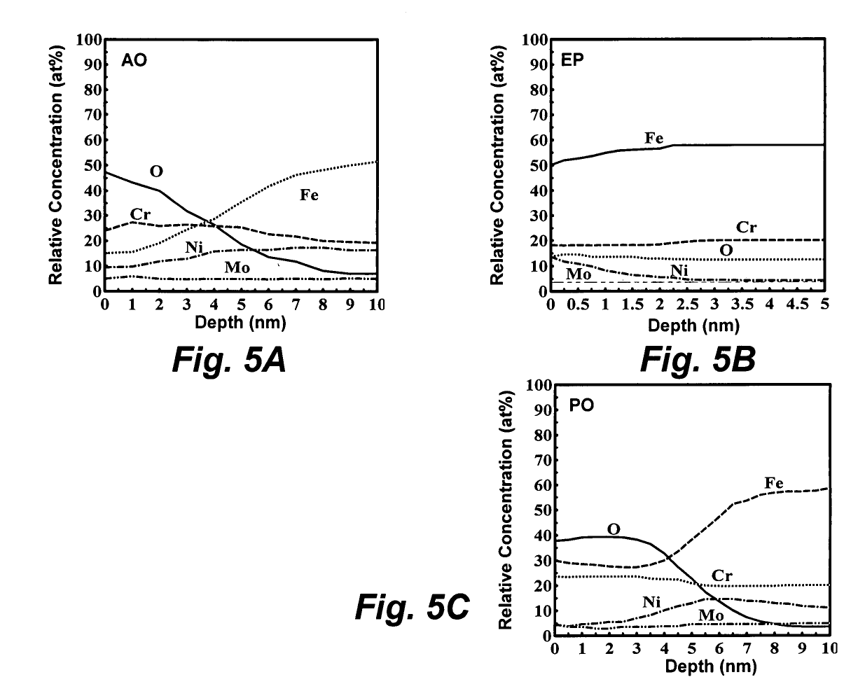

Referring now more specifically to FIGS. 5A-5C, illustrated therein are the oxygen and chromium concentration profiles inside AO, EP and PO films, as depicted via the AES depth profiles of oxygen, iron, chromium, and nickel from the passivated stainless steel surfaces/implants. Iron oxide is the dominant chemical composition on the EP and PO passivated stainless steel surface (FIGS. 5B and 5C, respectively), wherein oxygen-rich and chromium-rich profiles are the distinguished features for the AO passivated stainless steel (FIG. 5A).

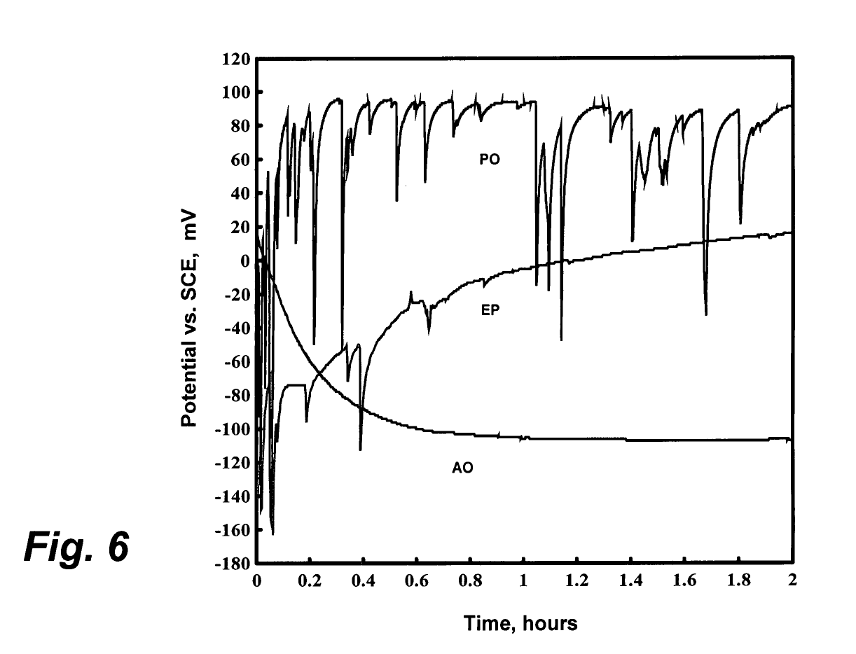

Referring now more specifically to FIG. 6, typical results of open-circuit potential (OCP) measurements on the AO, EP and PO oxide films are illustrated. Significant differences in OCP values are found for stainless steels passivated with AO, EP, and PO oxide film. AO treatment has the lowest OCP value (i.e., AO: −102±26 mV; PO: 100±41 mV; EP: 30±22 mV; P<0.01). The lower OCP for AO films is coincident with the suggestion within the art that an implant with an OCP close to the hydrogen potential could result in a thrombosis-free condition. Accordingly, because a blood vessel and blood possess negative charges in normal conditions, with the blood vessel having a negative potential, then a galvanic current could be induced when a metal with positive potential and charge is implanted within the vessel, and thus result in thrombosis.

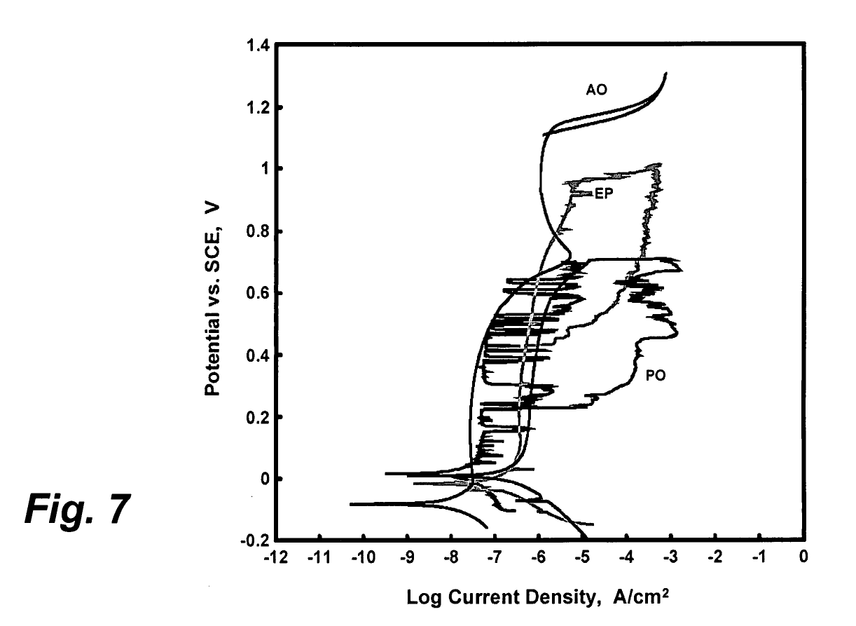

Referring now more specifically to FIG. 7, typical cyclic anodic polarization scanning curves for each surface condition of AO, EP and PO oxide films are illustrated. These scanning curves reveal an increase of the breakdown potential of treated samples ranging from 200 mV to 800 mV, except the stainless steel treated with AO. That is, no breakdown potential and low passive current density are found for AO treated stainless steel implants. It should be noted that the presence of a huge hysteresis loop is an indication of the nucleation and growth of corrosion pits. However, the small loop found on the AO treated stainless steel implant indicates that repassivation potential is higher than the Ecorr and represents the absence of any pitting degradation.

As may be illustrated with reference to FIG. 7, corrosion resistance of the surface treated stainless steel decreases in the order of AO>EP>PO according to the anodic polarization measurement. As such, it is apparent that stainless steel implants passivated with AO significantly improves the corrosion resistant performance of the implants.

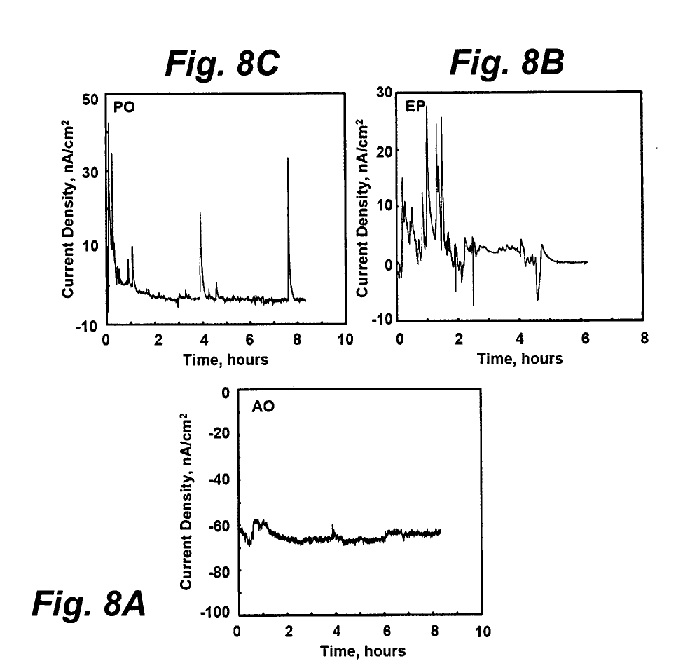

Referring now more specifically to FIGS. 8A-8C, current density at open-circuit potential (OCP) for the AO, EP and PO films are illustrated. Specifically, unsteady current densities, and spikes of current density, are detected for stainless steel implants passivated with PO and EP (see FIGS. 8A and 8B, respectively). However, consistent negative current density is obtained for the stainless steel implant passivated with AO surface film (see FIG. 8C).

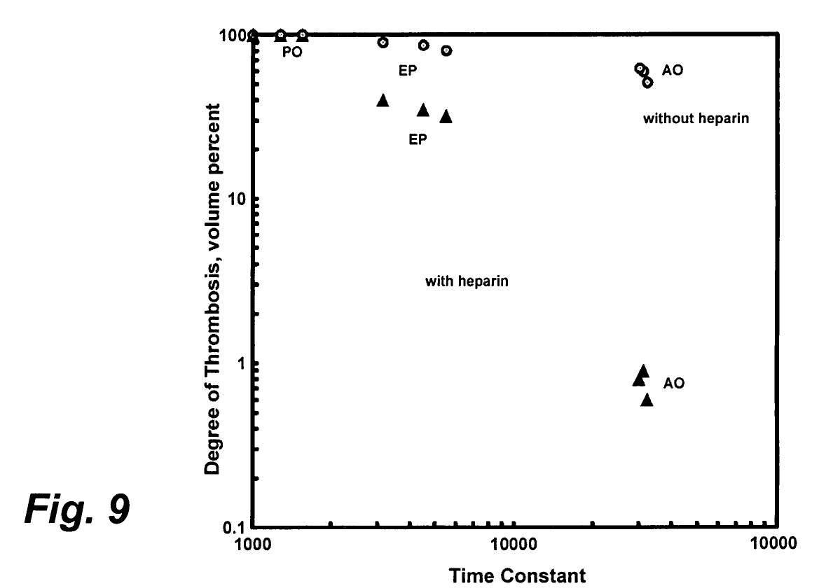

Referring now more specifically to FIG. 9, illustrated therein is the time constant (T_{c}) of the AO, EP and PO oxide films on implants, wherein the time constant is obtained by multiplying the values of resistance and capacitance from electrochemical impedance measurements. Specifically, implants with a higher value of time constant could result in a lower degree of thrombosis, wherein the difference becomes more significant when heparin is administrated in conjunction with implantation of the implant.

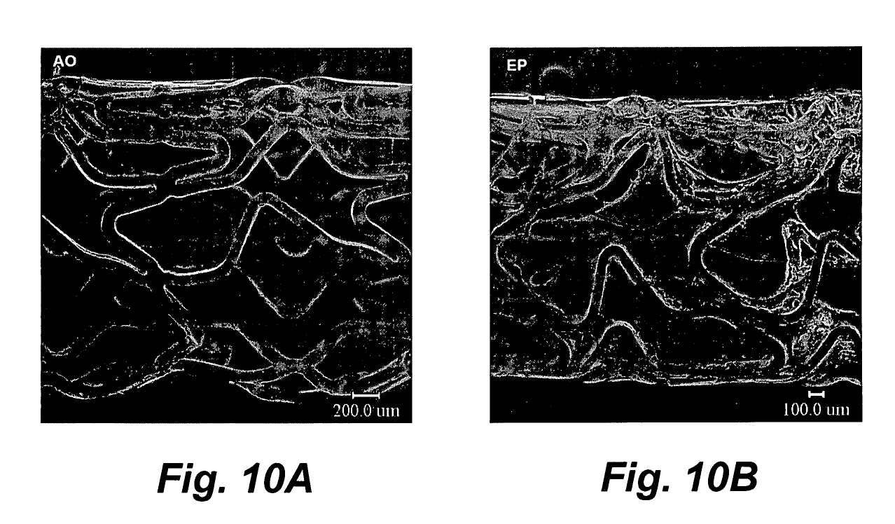

Referring now more specifically to FIGS. 10A and 10B, illustrated therein are implants with AO film and EP film, respectively, wherein a one-hour thrombosis study indicates that there is about a 90% reduction of thrombosis when heparin is utilized in conjunction with an implant metal with AO film, as opposed to the thrombotic condition developing with the EP film implant and heparin combination.

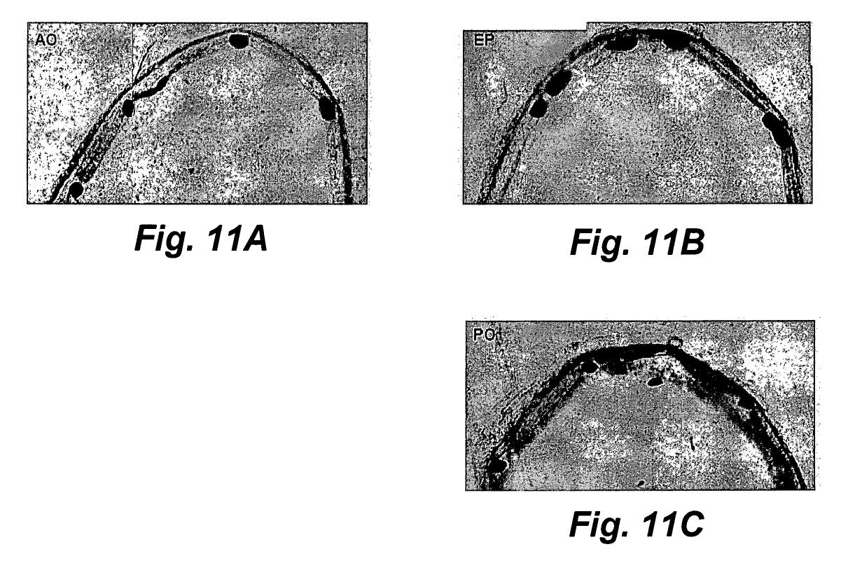

Referring now to FIGS. 11A-11C, illustrated therein is the effect of implants with AO, EP, and PO, respectively, on neointimal hyperplasia and ensuing lumen restenosis, as followed over an eight-week period. Neointimal hyperplasia is regarded as one of the major factors responsible for lumen restenosis following intravascular stent implantation. As depicted in respective FIGS. 11A and 11B, post-stenting neointima thickening was reduced by 50% for the stent group with AO film versus the stent group with an EP film.

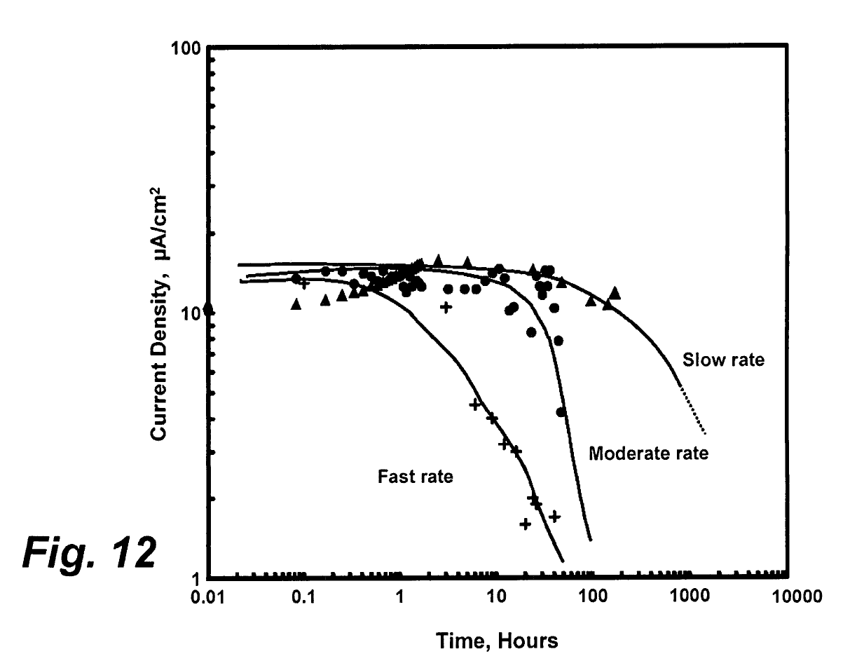

Referring now more specifically to FIG. 12, illustrated therein is the cyclic voltammetry of an AO film implant utilized as a platform for drug-coating. Specifically, the quantity of drugs on the implants can be determined by the cyclic voltammetry, and may be expressed as current density in a unit of μA/cm². Apparently, the eluting profile can be changed from a faster releasing rate to a slower eluting profile based upon the chemistries inside the AO layer. For an efficacious drug-eluting stent, the life-span must last at least 60 days after implantation of the stent to minimize the degree of neointima. Practically, the eluting profile for drugs (such as heparin, magnolol, tranilast, SIROLIMUS, TAXOL, and the like) from AO films, in-vitro, on stainless steel, can reach and even exceed the critical 60-day minimum requirement.

Having thus described exemplary embodiments of the present invention, it should be noted by those skilled in the art that the within disclosures are exemplary only, and that various other alternatives, adaptations, and modifications may be made within the scope of the present invention. Accordingly, the present invention is not limited to the specific embodiments illustrated herein, but is limited only by the following claims.

1. An amorphous oxide film, comprising:
a non-stoichiometric chromium oxide comprising a negative charge.

2. The amorphous oxide film of claim 1, further comprising ions selected from a group consisting of oxygen ions, chromium ions, hydroxyl ions, and combinations thereof.

3. An implantable device, comprising:
an amorphous oxide surface film comprising a negative charge thereover to prevent the release of positively-charged ions from said implantable device.

4. The implantable device of claim 3, wherein said amorphous oxide surface film comprises amorphous oxide particles on a nanometer to sub-nanometer scale.

5. The implantable device of claim 4, wherein said implantable device is heated within a saturated oxygen atmosphere such that said nanometer to sub-nanometer scale of said amorphous oxide particles form at a faster rate of nucleation than growth.

6. The implantable device of claim 5, wherein said saturated oxygen atmosphere comprises sodium nitrate.

7. The implantable device of claim 5, wherein said saturated oxygen atmosphere comprises a nitrate compound.

8. The implantable device of claim 5, wherein said saturated oxygen atmosphere comprises a nitrate compound selected from the group consisting of sodium nitrate, potassium nitrate, ammonium nitrate, calcium nitrate, chromium nitrate, copper nitrate, iron nitrate, lead nitrate, barium nitrate, and combinations thereof.

9. The implantable device of claim 5, wherein said saturated oxygen atmosphere comprises pH buffer chemicals selected from the group consisting of sodium bicarbonate, sodium carbonate, sodium hydroxide, and combinations thereof.

10. The implantable device of claim 5, wherein said saturated oxygen atmosphere comprises pH buffer chemicals selected from the group consisting of sodium bicarbonate, sodium carbonate, sodium hydroxide, phosphate compounds, borate compounds, and combinations thereof.

11. The implantable device of claim 5, wherein said nanometer to sub-nanometer scale of said amorphous oxide particles results in said amorphous oxide surface film comprising oxygen, chromium and hydroxyl ions therewithin to yield a non-stoichiometric chromium oxide comprising a negative charge.

12. The implantable device of claim 3, further comprising a thrombotic-reducing drug.

13. A passivation solution for facilitating the formation of an amorphous oxide layer over an implantable device, said solution comprising:
sodium nitrate as an oxygen provider to facilitate formation of said amorphous oxide layer over the implantable device.

14. The passivation solution of claim 13, further comprising pH buffer chemicals selected from the group consisting of sodium bicarbonate, sodium carbonate, sodium hydroxide, and combinations thereof, wherein said pH buffer chemicals functions as oxygen donors.

15. The passivation solution of claim 13, further comprising sodium bicarbonate, sodium carbonate and sodium hydroxide, as pH buffer chemicals, in a ratio of approximately 1:1:1.

16. The passivation solution of claim 13, further comprising sodium bicarbonate, sodium carbonate and sodium hydroxide, as pH buffer chemicals, in a ratio of approximately 1:1:10.

17. The passivation solution of claim 13, further comprising sodium bicarbonate, sodium carbonate and sodium hydroxide, as pH buffer chemicals, in a ratio of approximately 1:1:20.

18. The passivation solution of claim 13, further comprising nitric acid.

19. The passivation solution of claim 13, further comprising hydrochloric acid.

20. A method of forming an amorphous oxide surface film over an implantable device, said method comprising the step of:
a. creating a pH buffering solution comprising sodium hydroxide.

21. The method of claim 20, wherein said pH buffering solution further comprises sodium bicarbonate.

22. The method of claim 21, wherein said pH buffering solution further comprises sodium carbonate.

23. The method of claim 22, further comprising step b.: mixing said sodium bicarbonate, said sodium carbonate and said sodium hydroxide with distilled water.

24. The method of claim 23, further comprising step c.: agitating said sodium bicarbonate, said sodium carbonate and said sodium hydroxide with said distilled water until dissolved so as to bring said pH buffering solution to a pH of approximately around or higher than 10.

25. The method of claim 24, further comprising step d.: adding sodium nitrate to said pH buffering solution to yield a passivation solution.

26. The method of claim 25, further comprising step e.: heating said passivation solution to boiling.

27. The method of claim 26, further comprising step f.: adding the implantable device to the boiling said passivation solution to passivate the implantable device for a selected period of time, and thus facilitate the formation of said amorphous oxide surface film over the implantable device.

28. The method of claim 27, wherein said passivation solution comprises a composition of approximately 100 g/l of sodium nitrate, approximately 15 g/l of sodium bicarbonate, approximately 15 g/l of sodium carbonate, and approximately 15 g/l of sodium hydroxide.

29. The method of claim 27, wherein said passivation solution comprises sodium nitrate within a range of approximately 10-2000 g/l.

30. The method of claim 29, wherein said passivation solution comprises sodium bicarbonate within a range of approximately 0.1-50 g/l.

31. The method of claim 30, wherein said passivation solution comprises sodium carbonate within a range of approximately 0.1-50 g/l.

32. The method of claim 31, wherein said passivation solution comprises sodium hydroxide within a range of approximately 0.1-50 g/l.

33. A method of forming an amorphous oxide surface film over an implantable device, said method comprising the steps of:
a. creating a pH buffering solution comprising sodium hydroxide and water;
b. adding nitric acid to said pH buffering solution to yield a passivation solution.

34. The method of claim 33, wherein said passivation solution comprises a pH value of approximately 2 or lower.

35. The method of claim 34, wherein nitric acid solution comprises an approximately 1:1 ratio of concentrated nitric acid to water.

36. The method of claim 35, further comprising step c.: heating said passivation solution to boiling.

37. The method of claim 36, further comprising step d.: adding the implantable device to the boiling said passivation solution to passivate the implantable device for a selected period of time, and thus facilitate the formation of said amorphous oxide surface film over the implantable device.

38. The method of claim 37, wherein said passivation solution comprises a composition of approximately 1000 g/l of sodium nitrate.

39. The method of claim 37, wherein said passivation solution comprises sodium nitrate within a range of approximately 10-2000 g/l.

40. A method of forming an amorphous oxide surface film over an implantable device, said method comprising the steps of:
a. exposing the implantable device to a passivation solution comprising sodium nitrate, sodium bicarbonate and diluted hydrochloric acid, wherein said passivation solution comprises a pH value in a range of approximately 6.5 to approximately 7.5.

41. A method of forming an amorphous oxide surface film over an implantable device, said method comprising the steps of:
a. degreasing the implantable device;
b. pre-heating the implantable device;
c. pickling the implantable device;
d. passivating the implantable device within a passivation solution comprising sodium nitrate as an oxygen provider to facilitate formation of said amorphous oxide layer over the implantable device, and pH buffer chemicals selected from the group consisting of sodium bicarbonate, sodium carbonate, sodium hydroxide, and combinations thereof; and,
e. selectively rinsing the implantable device following any of said steps a-d.

42. The method of claim 41, further comprising step f: drying and sterilizing the implantable device.
